(19) United States
(12) Patent Application Publication
(10) Pub. No.: US 2009/0214569 A1
(43) Pub. Date: Aug. 27, 2009

(54) Use of HIV envelope/CD4 complexes for the generation antibodies

(75) Inventors: Jinhai Wang, Gaithersburg, MD (US); Michael Norcross, Bethesda, MD (US)

Correspondence Address: EDWARDS ANGELL PALMER & DODGE LLP, PO BOX 55874, BOSTON, MA, 02205, US

(73) Assignee: Government of the US, as represented by the Secretary, Dept. of Health & Human Services, Rockville, MD (US)

(21) Appl. No.: 11/990,953

(22) PCT Filed: Aug. 25, 2006
(86) PCT No.: PCT/US2006/033635

§ 371 Date: Feb. 24, 2009

Related U.S. Application Data
(60) Provisional application No. 60/711,985, filed on Aug. 25, 2005.

(51) Int. Cl. A61K 39/21; C07K 16/18; C12N 5/00

(52) U.S. Cl. 424/160.1; 530/387.9; 435/325; 424/208.1

(57) ABSTRACT

The instant invention provides antibodies, vaccines, and immunogenic compositions, for the treatment and prevention of HIV infection. The invention further provides kits comprising the antibodies, vaccines and immunogenic compositions, of the invention.

### RELATED APPLICATIONS

The instant application claims the benefit of U.S. Provisional Application No. 60/711,985, filed Aug. 25, 2005, the entire contents of which is expressly incorporated herein by reference.

### GOVERNMENT SUPPORT

Research supporting this application was carried out by the Government of the United States of America as represented by the Secretary, Department of Health and Human Services, and the government may have certain rights in this invention.

### BACKGROUND OF THE INVENTION

HIV infection has been implicated as the primary cause of the slowly degenerate disease of the immune system termed acquired immune deficiency syndrome (AIDS) (Barre-Sinoussi et al. (1983) Science 220:868-870; and Gallo et al. (1984) Science 224:500-503). Infection of the CD4+ subclass of T-lymphocytes with the HIV-1 virus leads to depletion of this essential lymphocyte subclass which inevitably leads to opportunistic infections, neurological disease, neoplastic growth and typically death. HIV-1 infection and HIV-1 associated diseases represent a major health problem and considerable attention is currently being directed towards the successful design of effective therapeutics.

HIV-1 is a member of the lentivirus family of retroviruses (Teich et al. (1984) In RNA Tumor Viruses ed. R. Weiss, N. Teich, H. Varmus, J. Coffin CSH Press, pp. 949-56). The life cycle of HIV-1 is characterized by a period of proviral latency followed by active replication of the virus. The primary cellular target for the infectious HIV-1 virus is the CD4 subset of human T-lymphocytes. Targeting of the virus to the CD4 subset of cells is due to the fact that the CD4 cell surface protein acts as the cellular receptor for the HIV-1 virus (Dalgleish et al. (1984) Nature 312:763-67; Klatzmann (1984) Nature 312:767-68; and Maddon et al. (1986) Cell 47:333-48).

After binding to the cell surface, the HIV-1 virion becomes internalized, and once inside the cell, the viral life cycle begins by conversion of the RNA genome into linear DNA molecules. This process is dependent on the action of the virally encoded reverse transcriptase. Following replication of the viral genome, the linear DNA molecule integrates into the host genome through the action of the viral integrase protein, thus establishing the proviral form of HIV-1.

HIV-1 utilizes several cell membrane proteins as its coreceptor to falitate viral entry into the host cell (Alkhatib et al. (1996) Science 272: 1955-1958; and Deng et al. (1996) Nature 388:296-300). Examples of chemokine receptors include CD4, CXCR4 and CCR5.

The CD4 molecule is the primary receptor for the human immunodeficiency virus type 1 (HIV-1) and is found predominantly on the surface of T-lymphocytes. The binding of HIV-1 to CD4 occurs via the major viral envelope glycoprotein gp120 and initiates the viral infection process. The HIV viral particle comprises a viral core, composed in part of capsid proteins, together with the viral RNA genome and those enzymes required for early replicative events. Myristylated gag protein forms an outer shell around the viral core, which is, in turn, surrounded by a lipid membrane envelope derived from the infected cell membrane. The HIV envelope surface glycoproteins are synthesized as a single 160 kilodalton precursor protein, which is cleaved by a cellular protease during viral budding into two glycoproteins, gp41 and gp120. gp41 is a transmembrane glycoprotein and gp120 is an extracellular glycoprotein, which remains non-covalently associated with gp4 1, in a trimeric or multimeric form.

A plethora of attempts to develop an effective HIV vaccine have been made, all of which have been unsuccessful. A number of strategies for developing vaccines against infection by the human immunodeficiency virus have focused on eliciting antibodies against the viral envelope glycoprotein gp120. There are several disadvantages to use gp120 as a vaccine. Although neutralizing antibodies against gp120 are elicited in vivo in HIV-1-infected individuals, hypervariability of the V3 epitope among different strains is a major obstacle for the generation of effective neutralizing antibodies against cross-clade strains of HIV-1. In most cases V3 loop antibodies neutralize HIV-1 in an isolate specific manner. Escape mutants develop after prolonged culturing of HIV-infected cells with type specific anti-V3 loop antibodies. Moreover, in most primary isolates the V3 loop is generally only a weak cross-neutralization epitope. The failure of gp120 as an immunogen by itself could be due to conformational masking of neutralizing epitopes inside the molecule. Other neutralizing epitopes encompassing genetically conserved regions of Env have been identified, many of which are discontinuous in nature. Some of these are strong neutralization epitopes for T-cell line-adapted viruses.

Further, CD4 binding site-related epitopes, encompassing a conserved region, elicit neutralizing antibodies against cross-clade isolates. However, this region is not normally an immunodominant epitope on the glycoprotein, and these antibodies do not effectively neutralize multiple primary isolates.

Due to the problems identified above, researchers have used a soluble complex of CD4 and gp120 for vaccine development. This approach has also been ineffective due the fact that soluble CD4 is a monomer and immunogenic.

Another approach has been to mix tumor cells expressing gp120 transgene and tumor cells expressing CD4 and CCR5 transgenes. The expression of gp120 transgene in the model is low, highly variable and unstable. The tightly formed viral synapses between env transfected cells and coreceptors transfected cells may bury most epitopes. Moreover; it is not clinically feasible to inject tumor cells into subjects for the treatment or prevention of HIV infection.

Lastly, it has been suggested that trimeric Env may be an effective immunogen to induce neutralizing antibody. However, current trimers demonstrated greatly reduced binding to CD4 as compared with monomer gp120.

Accordingly, the need exists for effective immunogens and methods of using these immunogens for the production of effective HIV vaccine.

### SUMMARY OF THE INVENTION

The instant invention is based, at least in part, on the discovery that complexes between cell surface proteins and viral envelope proteins present epitopes not present on the uncomplexed molecules. These epitopes elicit neutralizing antibodies with novel specificities and are thus useful in vaccines, immunogenic compositions, and/or immunotherapy of patients infected with, or at risk of being infected with, HIV. Further, these epitopes are found in regions of the virus conserved across HIV clades and therefore the antibodies they elicit will have broad reactivity against many different HIV strains.

Moreover, the methods of the instant invention induce epitopes specific to complexed gp120 that may not be normal targets for antibodies generated in vivo during infection. Although anti-CD4 antibodies can mediate cytotoxic effects, cell surface CD4, not like soluble CD4, is in its native conformation and it is not likely to induce anti-self antibodies. Antibodies against complex-specific epitopes are not expected to elicit anti-self antibodies capable of recognizing free cell surface CD4 on normal cells. Indeed, experimental results indicated that sera to immunogenic complexes of the invention does not cross react with CD4 on human PBLs. The complexes of the invention are stable without covalent cross-linking, and no cross-linking is planned because cross-linking may restrict the natural movement of the polypeptides relative to each other, thereby masking essential neutralizing epitopes.

Accordingly, in one aspect, the instant invention provides an antibody that specifically binds to a complex comprising one or more cell surface polypeptides, or a fragments thereof, expressed on a cell or cell line and one or more HIV envelope polypeptides, or a fragments thereof. In one embodiment, the complex is a non-covalent complex.

In a related embodiment, the complex presents one or more epitopes that are not presented in the absence of the non-covalent complex, i.e., a cryptic epitope such as, for example, 48d or 17b.

In another embodiment, the cell surface polypeptide is selected from CD4, CCR5, CXCR4. In related embodiments, the cell is a CD4⁺ cell, CD4⁺CCR5⁺ cell, CD4⁺CXCR4⁺ cell, or CD4⁺CCR5⁺CXCR4⁺ cell. In a specific embodiment, the cell is a CD4+ cell.

In another embodiment, the HIV envelope polypeptide is a glycoprotein, e.g., gp41, gp120, gp140, and gp160.

In yet another embodiment, the antibody is a neutralizing antibody.

In one specific embodiment, the cell surface polypeptide, or fragment thereof, is CCR5 and the HIV envelope polypeptide, or fragment thereof, is gp120. In another specific embodiment, the cell surface polypeptide, or fragment thereof, is CXCR4 and the HIV envelope polypeptide, or fragment thereof, is gp120. In another specific embodiment, the cell surface polypeptides, or fragment thereof, is CD4 and the HIV envelope polypeptide, or fragment thereof, is gp120.

In certain embodiment, the cell line is a mammalian cell or cell line.

In another aspect, the instant invention provides an immunogenic complex comprising a cell or cell line expressing one or more cell surface polypeptides, or a fragments thereof, having one or more HIV envelope polypeptides, or a fragments thereof, bound to the cell surface polypeptide.

In one embodiment, the complex is a non-covalent complex.

In one embodiment, the complex presents one or more epitopes that are not presented in the absence of the non-covalent complex.

In one embodiment, the cell surface polypeptide is selected from CD4, CCR5, CXCR4, or a combination thereof. In one embodiment the cell is a CD4+ cell.

In one embodiment, the HIV envelope polypeptide is a glycoprotein, e.g., gp41, gp120, gp140, or gp160.

In one specific embodiment, the cell surface polypeptide, or fragment thereof, is CCR5 and the HIV envelope polypeptide, or fragment thereof, is gp120. In another specific embodiment, the cell surface polypeptide, or fragment thereof, is CXCR4 and the HIV envelope polypeptide, or fragment thereof, is gp120. In another specific embodiment, the cell surface polypeptide, or fragment thereof, is CD4 and the HIV envelope polypeptide, or fragment thereof, is gp120.

In a specific aspect, the invention provides an immunogenic complex comprising a CD4+ mammalian cell expressing CCR5 and/or CXCR4, wherein gp120 is non-covalently bound to the CD4 and CCR5 and/or CXCR4. In a related embodiment, the complex presents one or more epitopes that are not presented by the cell in the absence of the complex.

In another aspect, the invention provides a vaccine, or immunogenic composition, comprising one or more of the immunogenic complexes described above. In related embodiment, the vaccine, or immunogenic composition, further comprises an adjuvant.

In another aspect, the invention provides a pharmaceutical composition comprising one or more of the immunogenic complexes described above, and a pharmaceutically acceptable carrier.

In another aspect, the invention provides an antigen, such as those described herein, for the generation of antibodies, especially neutralizing antibodies for the diagnosis and treatment of HIV-1 infection.

In another aspect, the invention provides a method of treating a subject having, or at risk of having HIV, by administering to the subject an effective amount of the vaccines, immunogenic compositions, or pharmaceutical compositions described herein, thereby treating the subject having, or at risk of having HIV.

In another aspect, the instant invention provides a method for producing an immune response to a HIV virus in a subject by administering to the subject an effective amount of the one or more of the vaccines, immunogenic compositions, or pharmaceutical compositions described herein, thereby producing an immune response to a HIV virus in a subject.

The invention further provides pharmaceutical compositions and kits containing the antibodies and compositions described herein.

### DESCRIPTION OF THE DRAWINGS

FIG. 1 depicts the binding of HIV-1 env to NIH3T3CD4 cells. In FIG. 1, the binding of env to cell surface CD4 was detected by FITC-labeled HIVIG. FITC-labeled IgG was used as negative control (Top frame). Top two panels were NIH3T3CD4 cells without gp120. The remaining four panels were NIH3T3CD4 cells incubated with gp120 from HIV-1 strains Ba-L, CM, FL, or HB10, respectively. The specificity was demonstrated by the competitive inhibition with sCD4 and no env was detected on NIH3T3 cells (not shown). NIH3T3CD4 cells were treated with enzyme free dissociation buffer for 5 minutes at 37° C., washed with cold PBS, blocked with 10% normal rabbit serum, then incubated with indicated gp120 for one hour at 4° C. After three washes with cold PBS, cells were incubated with FITC-labeled HIVIG at 4° C. for 30 minutes. Cells were subjected to flow cytometry analysis after three washes and fixation.

FIG. 2 depicts the detection of CD4 inducible epitope 48d on env-bound NIH3T3CD4 cells. NIH3T3CD4 cells were incubated with gp120 at 4° C. for one hour. Cells were washed three times and then incubated with 10% normal rabbit serum for thirty minutes. 48d monoclonal antibody or isotype control was added for one hour, followed by PE-labeled goat F(ab′)2 anti-mouse IgG(H+L) (Caltag) staining and cells were analyzed using flow cytometry.

FIG. 3 depicts the binding of PE-labeled anti-CD4 (Leu-3A) to NIH3T3 CD4 cells in the absence or presence of env. NIH3T3CD4 cells were incubated with indicated gp120 at 4° C. for 60 minutes, washed three times, then stained with PE-labeled anti-CD4 (Leu-3A, Becton Dickinson) and subjected to flow cytometry analysis.

FIG. 4 depicts the expression of CD4 and CCR5 in NIH3T3CD4 cells and NIH3T3CD4CCR5 cells. NIH3T3CD4 was stained positive with PE-labeled anti-CD4 (Leu-3A), but negative for CCR5. NIH3T3CD4CCR5 was stained positive with PE-labeled anti-CD4 (Leu-3A) and PE-labeled anti-CCR5 antibody 2D7 (BD Pharmingen).

FIG. 5 demonstrates that immune serum, but not control serum, specifically interacts with gp120 loaded PBLs, but not PBLs themselves. Gp120 loaded PBLs were incubated with diluted immune serum 1-1-HBCF (abbreviation of HB10, Ba-L, CM, FL), which was generated against NIH3T3CD4 cells loaded with gp120 mixture of HB10, Ba-L, CM, and FL strains, or negative control serum at 4° C. for 60 minutes. After three washes cells were stained with goat F(ab′)2 anti-mouse IgG(H+L) (Caltag) and analyzed on flow cytometry.

FIGS. 6A-B demonstrate competitive binding of 17b or anti-CD4 to env-loaded PBLs by immune serum. PBLs were loaded with or without env in the presence or absence of immune serum. The binding of FITC-labeled 17b was measured in FIG. 6A. CD4 staining was measured in FIG. 6B.

### DETAILED DESCRIPTION OF THE INVENTION

The instant invention is based, at least in part, on the discovery that complexes between cell surface proteins expressed on cells, e.g., mammalian cells, and viral envelope proteins present epitopes, i.e., cryptic epitopes, not present on the uncomplexed molecules. These epitopes elicit neutralizing antibodies with novel specificities and are thus useful in vaccines and/or immunotherapy of patients infected with, or at risk of being infected with, HIV.

Binding of the HIV-1 envelope glycoprotein (Env, gp120-gp41) to CD4 and coreceptors, e.g., CCR5 and/or CXCR4 initiates a series of conformational changes that are the heart of the fusion machinery leading to viral entry. The elucidation of the nature of the Env conformational changes is not only a clue to the mechanism of HIV type 1 (HIV-1) entry but may also provide new tools for the development of inhibitors and vaccines. It has been proposed that the interaction of coreceptor molecules with the Env-CD4 complex leads to intermediate Env conformations that may include structures conserved among various HIV-1 isolates that could be used as vaccines. Of the four known potent broadly neutralizing antibodies, none reacts with a receptor-inducible epitope.

It is an object of the present invention to provide antibodies to receptor-inducible epitopes, wherein the antibodies exhibit high affinity of binding of HIV and neutralizing activity. The antibodies can be used, alone or in combination with other active agents or as fusion proteins or conjugates with other active agents, to inhibit HIV and as tools to dissect mechanisms of HIV cellular entry. It is further an object of this invention to provide vaccines against HIV.

By “Env polypeptide” or “HIV envelope polypeptide” is meant a molecule derived from an HIV envelope protein. The envelope protein of HIV-1 is a glycoprotein of about 160 kd (gp160). As used herein, it is intended that the term HIV-1 envelope polypeptide refer to monomers or oligomers of the HIV-1 envelope polypeptide. During virus infection of the host cell, gp160 is cleaved by host cell proteases to form gp120 and the integral membrane protein, gp41. The gp41 portion is anchored in (and spans) the membrane bilayer of virion, while the gp120 segment protrudes into the surrounding environment. As there is no covalent attachment between gp120 and gp41, free gp120 is released from the surface of virions and infected cells. Env polypeptides may also include gp140 polypeptides. Env polypeptides can exist as monomers, dimers, trimers or multimers.

By a “gp120 polypeptide” is meant a molecule derived from a gp120 region of the Env polypeptide. Preferably, the gp120 polypeptide is derived from HIV Env. The primary amino acid sequence of gp120 is approximately 511 amino acids, with a polypeptide core of about 60,000 daltons. The polypeptide is extensively modified by N-linked glycosylation to increase the apparent molecular weight of the molecule to 120,000 daltons. The amino acid sequence of gp120 contains five relatively conserved domains interspersed with five hypervariable domains. The hypervariable domains contain extensive amino acid substitutions, insertions and deletions. Despite this variation, most, if not all, gp120 sequences preserve the virus's ability to bind to the viral receptor CD4. A “gp120 polypeptide” includes both single subunits or multimers.

Furthermore, an “Env polypeptide” or “gp120 polypeptide” as defined herein is not limited to a single polypeptide sequence. Indeed, the HIV genome is in a state of constant flux and contains several variable domains which exhibit relatively high degrees of variability between isolates. It is readily apparent that the terms encompass Env (e.g., gp120) polypeptides from any of the identified HIV isolates, as well as newly identified isolates, and subtypes of these isolates.

By the term “cell surface polypeptide” is meant a polypeptide expressed on the surface of a cell. In one embodiment, the cell surface polypeptide is CD4. In another embodiment, the cell surface polypeptide is capable of associating with CD4 and/or a viral coat protein, e.g., an Env protein. In certain embodiments, the cell surface protein participates in the steps required for viral infection of the cell. Preferred cell surface polypeptides include members of the CC or CXC chemokine receptor families, e.g., CCR5 and CXCR4.

Antibodies of the Invention

The instant invention provides antibodies that bind to complexes comprising one or more cell surface polypeptides, or a fragments thereof, expressed on a cell or cell line and one or more HIV envelope polypeptides, or a fragments thereof. In one embodiment, antibodies specifically recognize epitopes presented only when the complex is formed. In one embodiment, the complex is a non-covalent complex.

The term “isolated” refers to a molecule, e.g., an antibody, that is substantially free of its natural environment. For instance, an isolated antibody is substantially free of cellular material or other proteins from the cell or tissue source from which it is derived. The term “isolated” also refers to preparations where the isolated antibody is sufficiently pure to be administered as a pharmaceutical composition, or at least 70-80% (w/w) pure, more preferably, at least 80-90% (w/w) pure, even more preferably, 90-95% pure; and, most preferably, at least 95%, 96%, 97%, 98%, 99%, or 100% (w/w) pure.

As used herein, the term “antibody” refers to any molecule which has specific immunoreactivity activity, whether or not it is coupled with another compound such as a targeting agent, carrier, label, toxin, or drug. Although an antibody usually comprises two light and two heavy chains aggregated in a “Y” configuration with or without covalent linkage between them, the term is also meant to include any reactive fragment or fragments of the usual composition, such as Fab molecules, Fab proteins or single chain polypeptides having binding affinity for an antigen. Fab refers to antigen binding fragments. As used herein, the term “Fab molecules” refers to regions of antibody molecules which include the variable portions of the heavy chain and/or light chain and which exhibit binding activity. “Fab protein” includes aggregates of one heavy and one light chain (commonly known as Fab), as well as tetramers which correspond to the two branch segments of the antibody Y (commonly known as F(ab)₂), whether any of the above are covalently or non-covalently aggregated so long as the aggregation is capable of selectively reacting with a particular antigen or antigen family.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with the proteins disclosed herein. The antibodies can be tested for their desired activity using the in vitro assays described herein, or by analogous methods, after which their in vivo therapeutic and/or prophylactic activities are tested according to known clinical testing methods.

The term “neutralizing antibodies” as used herein refers to antibodies, or fragments thereof, that block a virus, e.g., HIV, from infecting a cell by, for example, blocking one or more receptors on the cell or the virus.

The term “specifically binds” as used herein refers to the affinity of an antibody, or fragment thereof, for a target epitope, e.g., an cryptic epitope presented in a complex comprising a cell surface polypeptide and a envelope polypeptide. In certain embodiments, antibodies that specifically bind to a target epitope bind with 2, 5, 10, 20, 50, 100, 500, 1000, or more times the affinity for which the bind a non-targeted eptitope. In further embodiments, antibodies that specifically bind to a target have detectable binding to only that target under a given set of binding conditions, e.g., salt, temperature, and/or pH conditions.

In specific embodiments, the invention provides antibodies specific for cryptic epitopes, i.e., epitopes presented by complexes of cell surface proteins, such as CD4, and viral Env proteins, such as gp120, that are not presented in the absence of complex formation. In related embodiments, these epitopes are only presented when the complex comprises a cell surface protein that is expressed on a cell, i.e., not a cell surface protein that is expressed as soluble polypeptide.

The antibodies of the instant invention are raised against complexes of cell surface proteins, e.g., CCR5, and viral envelope proteins, e.g., gp120. In one preferred embodiment, these complexes are formed while the cell surface protein is expressed on the cell surface. Accordingly, the ordinary skilled artisan would be able to select a cell or cell line that expresses the desired cell surface protein. In one embodiment, the cells used to form the complexes of the invention are CD4+ cells. The ordinary skilled artisan is capable of genetically engineering a cell to express a cell surface protein, such as CD4 or CCR5, using methods that are routine in the art. For example, one of skill in the art could modify a cell that does not produce the desired cell surface protein using molecular biology techniques that are available to one of skill in the art.

The cell comprising a cell surface polypeptide, e.g., CD4, complexed with a HIV envelope protein, e.g., gp120 is used as an immunogen to inject into an animal to produce antibodies of the invention.

The antibody can be a polyclonal, monoclonal, recombinant, e.g., a chimeric or humanized, fully human, non-human, e.g., murine, or single chain antibody. Chimeric, humanized, but most preferably, completely human antibodies are desirable for applications which include repeated administration, e.g., therapeutic treatment of human patients, and some diagnostic applications. In a related embodiment, the antibody can be coupled to a toxin.

Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240: 1041-1043; Liu et al. (1987) Proc. Natl. Acad. Sci. USA 84: 3439-3443; Liu et al. (1987) J. Immunol. 139: 3521-3526; Sun et al. (1987) Proc. Natl. Acad. Sci. USA 84: 214-218; Nishimura et al. (1987) Canc. Res. 47: 999-1005; Wood et al. (1985) Nature 314: 446-449; and Shaw et al. (1988) J. Natl. Cancer Inst. 80: 1553-1559); Morrison, S. L. (1985) Science 229: 1202-1207; Oi et al. (1986) BioTechniques 4: 214; Winter U.S. Pat. No. 5,225,539; Jones et al. (1986) Nature 321: 552-525; Verhoeyan et al. (1988) Science 239: 1534; and Beidler et al. (1988) J. Immunol. 141: 4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13: 65-93); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12: 899-903).

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat No. 4,816,567; and Morrison et al., Proc. Natl. Acad. Sci. USA 81: 6851-6855 (1984)).

The present monoclonal antibodies can be made using any procedure which produces monoclonal antibodies. For example, monoclonal antibodies of the invention can be prepared using hybridoma methods, such as those described by Kohler and Milstein, Nature, 256: 495 (1975). In a hybridoma method, a mouse or other appropriate host animal is typically immunized with an immunizing agent to elicit lymphocytes that produce antibodies that will specifically bind to the immunizing agent.

The monoclonal antibodies also can be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of antibodies). Libraries of antibodies or active antibody fragments also can be generated and screened using phage display techniques, e.g., as described in U.S. Pat. No. 5,804,440 to Burton et al. and U.S. Pat. No. 6,096,551 to Barbas et al and WO 03/097697 to Sur Der Mer.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in International Patent Application Publication No. WO 94/29348, published Dec. 22, 1994, and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-lining antigen.

As used herein, the term “antibody or fragments thereof” encompasses chimeric antibodies and hybrid antibodies, with dual or multiple antigen or epitope specificities, single chain antibodies and fragments, such as F(ab′)2, Fab′, Fab, scFv and the like, including hybrid fragments. Thus, fragments of the antibodies that retain the ability to bind their specific antigens are provided. For example, fragments of antibodies which maintain HIV gp120 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York (1988)). Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase bio-longevity, to alter secretory characteristics; etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment (Zoller, M. J. Curr. Opin. Biotechnol. 3: 348-354 (1992)).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

Human antibodies also can be prepared using any other technique. Examples of techniques for human monoclonal antibody production include those described by Cole et al. (Monoclonal Antibodies and Cancer Therapy, Alan R. Liss, p. 77 (1985)) and by Boerner et al. (J. Immunol. 147(1): 86-95 (1991)). Human antibodies (and fragments thereof) also can be produced using phage display libraries (Hoogenboom et al., J. Mol. Biol. 227: 381 (1991); Marks et al., J. Mol. Biol. 222: 581 (1991)).

Human antibodies also can be obtained from transgenic animals. For example, transgenic, mutant mice that can produce a full repertoire of human antibodies in response to immunization have been described (see, e.g., Jakobovits et al., Proc. Natl. Acad Sci. USA 90: 2551-255 (1993); Jakobovits et al., Nature 362: 255-258 (1993); and Bruggermann et al., Year in Immunol. 7: 33 (1993)). Specifically, the homozygous deletion of the antibody heavy chain joining region (J(H) gene in these chimeric and germ-line mutant mice results in complete inhibition of endogenous antibody production, and the successful transfer of the human germ-line antibody gene array into such germ-line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature 321: 522-525 (1986); Reichmann et al., Nature 332: 323-327 (1988); and Presta, Curr. Opin. Struct. Biol. 2: 593-596 (1992)).

Methods for humanizing non-human antibodies are well-known in the art. For example, humanized antibodies can be generated according to the methods of Winter and co-workers (Jones et al., Nature 321: 522-525 (1986); Riechmann et al., Nature 332: 323-327 (1988); and Verhoeyen et al., Science 239: 1534536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

A fusion protein or conjugate (conjugate produced by chemical or physical means) comprising an above-described antibody is also provided. The fusion protein or conjugate can comprise an other antibody that binds to an epitope described herein, wherein the epitope recognized by the antibody is inducible, and wherein the antibody binding to the epitope is enhanced by the presence of a cell surface protein expressed on a cell and the HIV viral envelope polypeptide. As another alternative, the fusion protein or conjugate can comprise a toxin.

Toxins are poisonous substances produced by plants, animals, or microorganisms that, in sufficient dose, are often lethal. A preferred toxin is Pseudomonas toxin. Diphtheria toxin is a substance produced by Corynebacterium diphtheria, which can be used therapeutically. This toxin consists of an a subunit and a β subunit, which, under proper conditions, can be separated. Another example of a toxin is tetanus toxoid, which is produced by Clostridium tetani. Lectins are proteins, usually isolated from plant material, which bind to specific sugar moieties. Many lectins are also able to agglutinate cells and stimulate lymphocytes. However, ricin is a toxic lectin, which has been used immunotherapeutically. This is accomplished by binding the alpha-peptide chain of ricin, which is responsible for toxicity, to the antibody molecule to enable site-specific delivery of the toxic effect. Other therapeutic agents, which can be coupled to the antibodies, are known, or can be easily ascertained, by those of ordinary skill in the art.

The ordinary skilled artisan using the teachings above and the knowledge available in the art could make the antibodies of the invention using only routine experimentation.

Pharmaceutical Compositions and Kits

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt, gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar, buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an antibody or complex of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment, and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats HIV infection, or associated disease.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition. Moreover, the pharmaceutical compositions described herein may be administered with one or more other active ingredients that would aid in treating a subject having a HIV infection. In a related embodiment, the pharmaceutical compositions of the invention may be formulated to contain one or more additional active ingredients that would aid in treating a subject having a HIV infection or associated disease or disorder.

The antibodies and complexes, produced as described above, can be provided in kits, with suitable instructions and other necessary reagents, in order to conduct immunoassays as described above. The kit can also contain, depending on the particular immunoassay used, suitable labels and other packaged reagents and materials (i.e. wash buffers and the like). Standard immunoassays, such as those described above, can be conducted using these kits. The pharmaceutical compositions can be included in a container, pack, kit or dispenser together with instructions, e.g., written instructions, for administration, particularly such instructions for use of the antibody or complex to treat or prevent an HIV infection or associated disease. The container, pack, kit or dispenser may also contain, for example, one or more additional active ingredients that would aid in treating a subject having an HIV infection.

Methods of Treatment

The present invention provides for both prophylactic and therapeutic methods of treating a subject having, or at risk of having, an HIV infection.

As used herein, an “HIV-infection” means having at least cell invaded by HIV. Subjects with an increased risk of having an HIV infection include, for example, intravenous drug users and people with multiple sexual partners.

The instant invention further provides a method of treating an HIV-infected subject, which comprises administering to the subject one or more doses of a pharmaceutical composition of the invention effective to reduce the population of HIV-infected cells in the HIV-infected subject, thereby treating the HIV-infected subject.

The instant invention further provides a method of reducing the likelihood of an HIV-exposed subject's becoming infected with HIV, which comprises administering to the HIV-exposed subject a dose of the composition, e.g., the pharmaceutical composition, of the instant invention effective to reduce the population of HIV in the HIV-exposed subject, thereby reducing the likelihood of the subject's becoming infected with HIV.

The vaccines and pharmaceutical compositions of the invention may also ameliorate the progression of an HIV-related disorder in a subject to whom the vaccines or pharmaceutical compositions were administered while the subject was either non-HIV-exposed or HIV-exposed, but not yet HIV-infected.

The instant invention provides a method of reducing the likelihood of a non-HIV-exposed subject's becoming infected with HIV as a result of exposure thereto during an incident wherein there is an increased risk of exposure to HIV, which comprises administering to the subject immediately prior to the incident a dose of the composition of the subject invention effective to reduce the population of HIV to which the subject is exposed during the incident, thereby reducing the likelihood of the subject's becoming infected with HIV.

As used herein, the term “HIV-related disease” refers to both the asymptomatic and symptomatic phases, that is the ARC and AIDS phase, which follow HIV infection. The terms “AIDS” and “ARC” refer to Acquired Immune Deficiency Syndrome and AIDS-Related complex, respectively, as described by Adler, British Medicine, 294:1145 (1987), which is herein incorporated by reference. AIDS is characterized by tumors and a series of opportunistic infections. Moreover, one of skill in the art will recognize that diseases caused by related viruses, e.g., SIV or other retroviral diseases, are subject to treatment using the methods and compositions of the instant invention.

As used herein, the term “treatment” is defined as the application or administration of a therapeutic agent to a patient, or application or administration of a therapeutic agent to an isolated tissue or cell line from a patient, who has, or is at risk of having, an HIV infection, with the purpose to cure, heal, alleviate, relieve, alter, remedy, ameliorate, improve or affect the infection or the symptoms of infection. A therapeutic agent includes, but is not limited to, antibodies, or fragments thereof, or the complexes, e.g., cellular complexes, described herein.

The term “effective amount” refers to a dosage or amount that is sufficient to reduce the amount of HIV to result in amelioration of symptoms in a patient or to achieve a desired biological outcome, e.g., lower HIV titre.

The invention also contemplates a vaccine or immunogenic composition, for stimulating a host's immune system, comprising the complexes described herein. The vaccine, or immunogenic composition, optionally further comprises one or more pharmaceutically acceptable carriers, adjuvants and/or diluents.

As used herein, a “vaccine”, “immunogenic composition”, “cellular composition”, “cellular vaccine” or “cellular immunogen” refers to a composition comprising at least one cell population expressing a cell surface polypeptide complexed to an HIV envelope polypeptide, which is optionally inactivated, as an active ingredient.

The instant invention also provides vaccine compositions for use in vaccinating subjects against HIV. In one embodiment, the vaccine comprises a cultured mammalian cell. Cultured cells expressing a cell surface polypeptide, e.g., CD4, in complex with a HIV envelope polypeptide, e.g., gp120, according to the invention may be used in the preparation of vaccines. Such preparation uses routine methods known to persons skilled in the art. Typically, such vaccines are prepared as injectables, either as liquid solutions or suspensions; solid forms suitable for solution in, or suspension in, liquid prior to injection may also be prepared. The preparation may also be emulsified. The antibodies and complexes, e.g., non-covalent complexes, can be used in vaccine compositions, individually or in combination, in e.g., prophylactic (i.e., to prevent infection) or therapeutic (to treat HIV following infection) vaccines. The vaccines can comprise mixtures of one or more of the complexes described herein. The vaccine may also be administered in conjunction with other antigens and immunoregulatory agents, for example, immunoglobulins, CpG or non-CpG oligodeoxynucleotides, cytokines, lymphokines, and chemokines, including but not limited to IL-2, modified IL-2 (cys125-ser125), GM-CSF, IL-12, γ-interferon, IP-10, MIP1β and RANTES. The vaccines may be administered as polypeptides or using vectors (e.g., liposomes, particles coated with nucleic acid or protein). The vaccine may be given more than once (e.g., a “prime” administration followed by one or more “boosts”) to achieve the desired effects. The same composition can be administered as the prime and as the one or more boosts. Alternatively, different compositions can be used for priming and boosting.

The vaccines will generally include one or more “pharmaceutically acceptable excipients or vehicles” such as water, saline, glycerol, ethanol, etc. Additionally, auxiliary substances, such as wetting or emulsifying agents, pH buffering substances, and the like, may be present in such vehicles.

A carrier is optionally present which is a molecule that does not itself induce the production of antibodies harmful to the individual receiving the composition. Suitable carriers are typically large, slowly metabolized macromolecules such as proteins, polysaccharides, polylactic acids, polyglycollic acids, polymeric amino acids, amino acid copolymers, lipid aggregates (such as oil droplets or liposomes), and inactive virus particles. Such carriers are well known to those of ordinary skill in the art. Furthermore, the Env polypeptide may be conjugated to a bacterial toxoid, such as toxoid from diphtheria, tetanus, cholera, etc.

Adjuvants may also be used to enhance the effectiveness of the vaccines. Such adjuvants include, but are not limited to: (1) aluminum salts (alum), such as aluminum hydroxide, aluminum phosphate, aluminum sulfate, etc.; (2) oil-in-water emulsion formulations (with or without other specific immunostimulating agents such as muramyl peptides (see below) or bacterial cell wall components), such as for example (a) MF59 (International Publication No. WO 90/14837), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi.™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox.™); (3) saponin adjuvants, such as Stimulon.™ (Cambridge Bioscience, Worcester, Mass.) may be used or particle generated therefrom such as ISCOMs (immunostimulating complexes); (4) Complete Freunds Adjuvant (CFA) and Incomplete Freunds Adjuvant (IFA); (5) cytokines, such as interleukins (IL-1, IL-2, etc.), macrophage colony stimulating factor (M-CSF), tumor necrosis factor (TNF), etc.; (6) detoxified mutants of a bacterial ADβ-ribosylating toxin such as a cholera toxin (CT), a pertussis toxin (PT), or an E. coli heat-labile toxin (LIT), particularly LT-K63 (where lysine is substituted for the wild-type amino acid at position 63) LT-R72 (where arginine is substituted for the wild-type amino acid at position 72), CT-S 109 (where serine is substituted for the wild-type amino acid at position 109), and PT-K9/G129 (where lysine is substituted for the wild-type amino acid at position 9 and glycine substituted at position 129) (see, e.g., International Publication Nos. W093/13202 and W092/19265); and (7) other substances that act as immunostimulating agents to enhance the effectiveness of the composition.

Muramyl peptides include, but are not limited to, N-acetyl-muramyl-L-threonyl-D-isoglutamine (thr-MDP), N-acteyl-normuramyl-L-alanyl-D-isogluatme (nor-MDP), N-acetylmuramyl-L-alanyl-D-isogluatminyl-L-alanine-2-(1′-2′-dipalmitoyl-sn-glycero-3-hydroxyphosphoryloxy)-ethylamine (MTP-PE), etc.

Typically, the vaccine compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above.

The vaccines will comprise a prophylactically or therapeutically effective amount of the above identified antibodies or complexes, as needed. By “prophylactically or therapeutically effective amount” is meant an amount which will induce a protective immunological response in the uninfected, infected or unexposed individual to which it is administered. Such a response will generally result in the development in the subject of a secretory, cellular and/or antibody-mediated immune response to the vaccine. Usually, such a response includes but is not limited to one or more of the following effects; the production of antibodies from any of the immunological classes, such as immunoglobulins A, D, E, G or M; the proliferation of B and T lymphocytes; the provision of activation, growth and differentiation signals to immunological cells; expansion of helper T cell, suppressor T cell, and/or cytotoxic T cell.

Preferably, the effective amount is sufficient to bring about treatment or prevention of disease symptoms. The exact amount necessary will vary depending on the subject being treated; the age and general condition of the individual to be treated; the capacity of the individual's immune system to synthesize antibodies; the degree of protection desired; the severity of the condition being treated; the particular complex selected and its mode of administration, among other factors. An appropriate effective amount can be readily determined by one of skill in the art. A “therapeutically effective amount” will fall in a relatively broad range that can be determined through routine trials.

Vaccines can be administered either subcutaneously, epidermally, intradermally, intramucosally such as nasally, rectally and vaginally, intraperitoneally, intravenously, orally or intramuscularly. Other modes of administration include oral and pulmonary administration, suppositories, needle-less injection, transcutaneous and transdermal applications. Dosage treatment may be a single dose schedule or a multiple dose schedule. Administration of the vaccine may also be combined with administration of peptides or other substances.

### EXAMPLES

It should be appreciated that the invention should not be construed to be limited to the examples that are now described; rather, the invention should be construed to include any and all applications provided herein and all equivalent variations within the skill of the ordinary artisan.

Several studies were conducted to show that CD4 inducible epitopes on gp 120 could be exposed and the exposure of CD4 was dramatically masked by gp120. The studies also demonstrated that in mice after immunization with gp120-cell surface CD4 immunogen, neutralizing antibodies could be raised against gp120 or gp120/CD4 complexes, but not cell surface human CD4.

### Example 1

### Formation of Gp120-CD4 Complex on Cell Surface

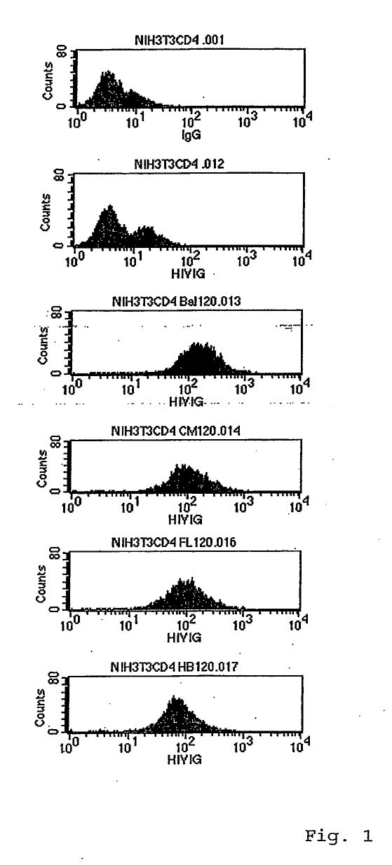

NIH 3T3CD4 or NIH 3T3CD4CCR5 stable cell lines established by Dr. Dan Littman (from NIAID AIDS Reagent Program) were cultured in DMEM with 10% fetal bovine serum. Cells were lifted by enzyme free medium that does not affect cell surface protein, washed with 1×PBS, pH7.4 three times. gp120 was detected on NIH 3T3 CD4 cell surface, but not on NIH 3T3 cell surface with HIVIG on flow cytometry analysis (FIG. 1). Gp120 binding to NIH 3T3CD4 cells was reduced in the presence of sCD4 (data not shown).

### Example 2

### Inducing CD4 Inducible Epitopes on gp120 by Cell Surface CD4

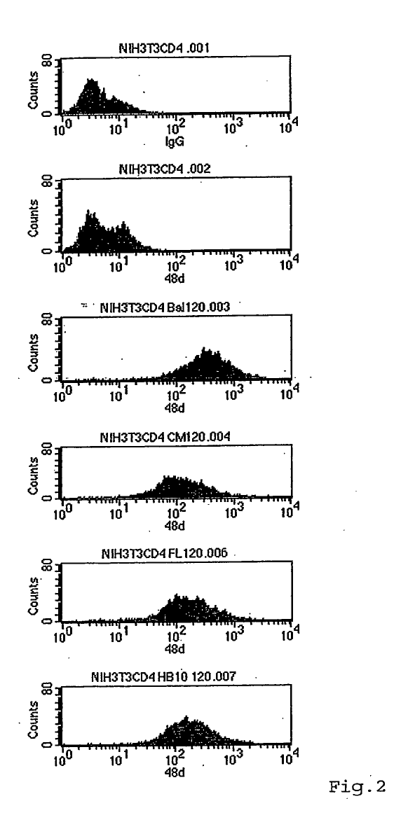

The following experiment was performed to determine whether it was possible to induce CD4 inducible epitopes on gp120 by cell surface CD4. The results demonstrate that the CD4 inducible epitope was detected on cell surface of NIH3T3CD4 loaded with gp120 by Monoclonal antibody 48d (FIG. 2).

### Example 3

### Masking CD4 Epitopes by Env

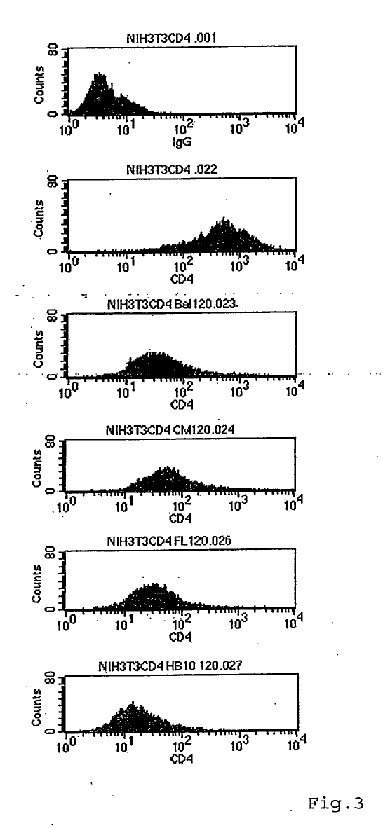

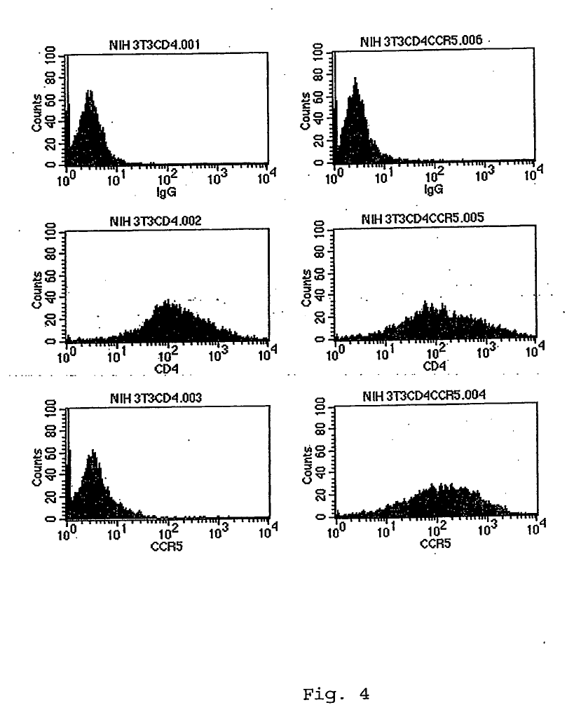

The following experiment was performed to determine whether CD4 was blocked by Env proteins on the cell surface. The experiments demonstrate that detection of CD4 by anti-CD4 antibody (Leu-3a) was dramatically blocked by Env on NIH3T3CD4 cells (FIGS. 3 and 4).

### Example 4

### Generation of Env Specific Sera

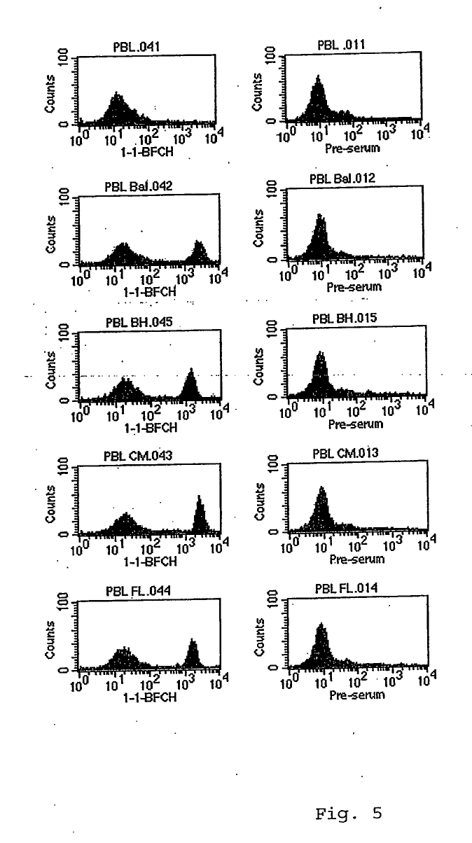

NIH3T3CD4-gp120 or NIH3T3CD4CCR5-gp120 in MPL+TDM emulsion were administered in both thighs (100 μl each site) of human CD4 and CCR5 transgenic mice (kindly supplied by Dr. Dan Littman). MIH3T3CD4-gp120 or NIH3T3CD4CCR5-gp120 was generated by incubating NIH3T3CD4 or NIH3T3CD4CCR5 cells (20×10⁶) with gp120 mixtures (5 μg each of HB10, Ba-L, CM, and FL strains) at 4° C. for one hour. After 3 immunizations administered three to four week apart, sera were collected from a group of five mice between 14 and 21 days after final boost using microtainer brand serum separator tube from Becton Dickinson. Sera from unimmunized mice were collected as control. The specificity of immune sera was tested with gp120 loaded or unloaded PBLs in flow cytometry analysis. As shown in FIG. 5, vaccine induced sera, not normal mouse sera react specifically with HIV env on primary PBLs. Neither vaccine induced sera, nor normal mouse sera reacted with PBLs. The titer of the generated immune sera was very high. Vaccine sera at 1:3200 still detected similar percentage of env-loaded PBLs.

### Example 5

### Cross-Clade Neutralization of HIV-1 Isolates

In order to best mimic the in vivo scenario, a simple culture method was established for in vitro neutralizing assay in the absence of mitogen. In this culture system, human peripheral blood lymphocytes (PBLs) were isolated from individual healthy donors by counter-current centrifugal elutriation. Elutriated PBLs composed of 90% CD3⁺ cells, 3-5% CD14⁺ monocytes, and 5% CD19⁺ B cells were cultured in RPMI 1640 medium supplemented with 10% fetal bovine serum (HyClone). After culture for 8 days, PBLs were pellet and resuspended in medium (2×10⁵ cells/100 μl) and added into round bottom 96-well plates contains immune or control sera, then exposed to different isolates at a dose equivalent to a p24 concentration of 25 ng ml⁻¹. After extensive washing with phosphate-buffered saline, PBLs were cultured in medium containing rhIL-2 (150 U ml⁻¹) for additional 8 days. Culture supernatants were collected and assayed for p24 by an enzyme immunoassay (EIA) (Coulter Corporation, Hialeah, Florida) and quantitated at 650 nm on a V max kinetic microplate reader (Molecular Devices, Sunnyvale, Calif.).

### Example 6

### Binding of 17b or Anti-CD4 to Env-Loaded PBLs

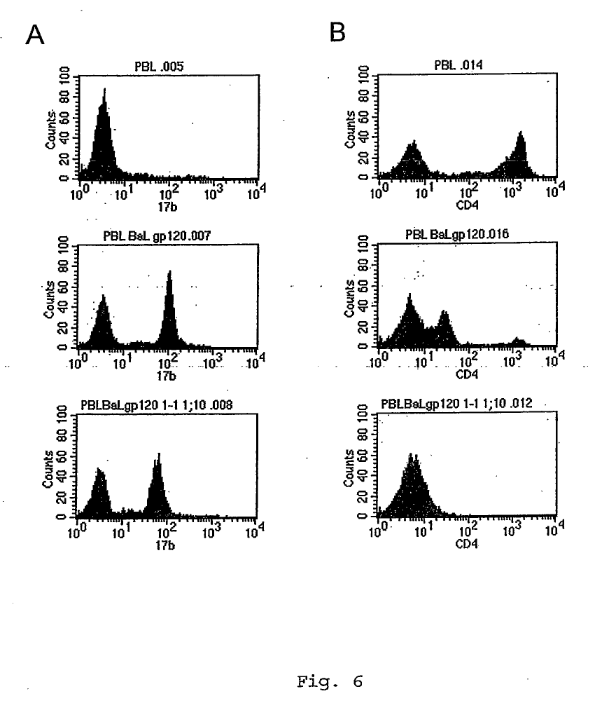

PBLs were loaded with or with out Env in the presence or absence of immune serum. The biding of FITC-labeled 17b was measured and is shown in FIG. 6. The data demonstrates the competitive binding of immune sera with 17b on Env loaded PBLs.

The mean fluorescence intensity (MFI) of 17b positive peak is 107 in samples without immune sera and the MFI is 71 in samples treated with 1 to 10 diluted immune sera 1-1-HBCF.

### Example 7

### Neutralizing of HIV-1 Isolates

Table 1 and 2 show the neutralizing of HIV-1 isolates of clade A, B, and C of group M. Human primary PBLs were cultured in RPMI-1640 containing 10% fetal bovine serum for 8 days, then infected with HIV-1 isolates in the presence of diluted immune serum or control serum overnight. After three washes with PBS cells were cultured in IL-2 containing complete medium for additional 8 days. At day 8 postinfection supernatant was assayed for p24 production. Immune serum 1-1-HBCF was generated against NIH3T3 CD4+cells loaded with gp120 mixture of B10, Ba-L, CM, and FL strains, then mixed in MPL+TDM emulsion (RIBI adjuvant system, Corixa). Immune serum 2-2-HBCF was generated against NIH3T3 CD4⁺ CCR5⁺ cells loaded with gp120 mixture of HB10, Ba-L, CM, and FL strains then mixed in MPL+TDM emulsion. HBCF was generated against gp120 mixture of HB10, Ba-L, CM, and FL strains in MPL+TDM emulsion. CD4CCR5 transgenic mice (from Dr. Dan Littman) were used for the immunization. All sera were heat-inactivated and used at 1:50 dilution.

TABLE 1

| | | | p24 | Inhibition |
|---|---|---|---|---|
| Strain | clade | treatment | pg/ml | (%) |
| 93BR012 | B, R5 | PS | 291658 | 0 |
| | | 1-1-HBCF | 15516 | 95 |
| | | 2-2-HBCF | 13271 | 95 |
| 92UG029 | A, X4 | PS | 50059 | 0 |
| | | 1-1-HBCF | 4067 | 92 |
| | | 2-2-HBCF | 49473 | 1 |
| 93UGA089 | A, R5 | PS | 71823 | 0 |
| | | 1-1-HBCF | 8082 | 89 |
| | | 2-2-HBCF | 67716 | 6 |

TABLE 2

| | | | | Inhibition |
|---|---|---|---|---|
| Strain | clade | treatment | p24 pg/ml | (%) |
| 93BR012 | B, R5 | PS | 73293 | 0 |
| | | 1-1-HBCF | <1000 | 99 |
| | | 2-2-HBCF | 4656 | 94 |
| | | HBCF | 57671 | 21 |
| 92UG029 | A, X4 | PS | 14686 | 0 |
| | | 1-1-HBCF | 7186 | 51 |
| | | 2-2-HBCF | <1000 | 99 |
| 93UG089 | A, R5 | PS | 17983 | 0 |
| | | 1-1-HBCF | <1000 | 99 |
| | | 2-2-HBCF | <1000 | 99 |
| CN006 | C, R5 | 1S | 249000 | 0 |
| | | 1-1-HBCF | 18084 | 93 |
| | | 2-2-HBCF | 183000 | 26 |
| US723 | B, R5X4 | PS | 1600000 | 0 |
| | | 1-1-HBCF | 455000 | 72 |
| | | 2-2-HBCF | 2010000 | −25 |
| | | HBCF | 1510000 | 6 |

### Incorporation by Reference

The contents of all references, patents, pending patent applications and published patents, cited throughout this application are hereby expressly incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

1. An antibody, or fragment thereof, that specifically binds to a complex comprising one or more cell surface polypeptides, or a fragments thereof, expressed on a cell or cell line and one or more HIV envelope polypeptides, or a fragments thereof.

2. The antibody, or fragment thereof, of claim 1, wherein the complex comprising one or more cell surface polypeptides, or a fragments thereof and one or more HIV envelope polypeptides, or a fragments thereof, is a non-covalent complex.

3. The antibody, or fragment thereof, of claim 2, wherein the complex comprising one or more cell surface polypeptides, or a fragments thereof and one or more HIV envelope polypeptides, or a fragments thereof, presents one or more epitopes that are not presented in the absence of the non-covalent complex.

4. The antibody, or fragment thereof, of claim 3, wherein said epitope is selected from the group consisting of the glycoprotein epitopes 48d and 1 7b.

5. The antibody, or fragment thereof, of claim 2, wherein the cell surface polypeptide is selected from CD4, CCR5, CXCR4, or any combination comprising CD4.

6. The antibody, or fragment thereof, of claim 1, wherein the cell is a CD4+ cell.

7. The antibody, or fragment thereof, of claim 1, wherein the HIV envelope polypeptide is a glycoprotein.

8. The antibody, or fragment thereof, of claim 7, wherein the glycoprotein is selected from the group consisting of gp120, gp140, and gp160.

9. The antibody, or fragment thereof, of claim 1, wherein the antibody is a neutralizing antibody.

10. The antibody, or fragment thereof, of claim 3, wherein the cell surface polypeptide, or fragment thereof, is CCR5 and the HIV envelope polypeptide, or fragment thereof, is gp120.

11. The antibody, or fragment thereof, of claim 3, wherein the cell surface polypeptide, or fragment thereof, is CXCR4 and the HIV envelope polypeptide, or fragment thereof, is gp120.

12. The antibody, or fragment thereof, of claim 3, wherein the cell surface polypeptide, or fragment thereof, is CD4 and the HIV envelope polypeptide, or fragment thereof, is gp120.

13. The antibody, or fragment thereof, of claim 1, wherein the cell or cell line is a mammalian cell or cell line.

14. A immunogenic complex comprising a cell or cell line expressing one or more cell surface polypeptides, or a fragments thereof, or liposomes and other particles containing one or more cell surface polypeptides, or a fragments thereof, and one or more HIV envelope polypeptides, or a fragments thereof, bound to the cell surface polypeptide.

15-23. (canceled)

24. An immunogenic complex comprising a CD4+ mammalian cell expressing CCR5 and/or CXCR4, wherein gp120 is non-covalently bound to the CD4 and CCR5 and/or CXCR4.

25. The immunogenic complex of claim 24, wherein the complex presents one or more epitopes that are not presented by the cell in the absence of the complex.

26. A vaccine, or immunogenic composition, comprising the antibody, or fragment thereof, of claim 1.

27. The vaccine, or immunogenic composition, of claim 26, wherein the vaccine is a cellular vaccine.

28. A pharmaceutical composition comprising the antibody of claim 1, and a pharmaceutically acceptable carrier.

29. The composition of any one of claims 28, further comprising an adjuvant.

30. A method of treating a subject having, or at risk of having HIV, comprising:
administering to the subject an effective amount of the pharmaceutical composition of claim 28;
thereby treating the subject having, or at risk of having HIV.

31. A method for producing an immune response to a HIV virus in a subject comprising;
administering to the subject an effective amount of the pharmaceutical composition of claim 28;
thereby producing an immune response to a HIV virus in a subject.
